(19) United States
(12) Patent Application Publication
(10) Pub. No.: US 2004/0002483 A1
(43) Pub. Date: Jan. 1, 2004

(54) Use of ester and ether derivatives of 4-hydroxy-19-norandrosterone to increase the level of the anabolic/androgenic hormone 4-hydroxy-19-nortestosterone in humans

(75) Inventor: Sal Abraham, Dunmore, PA (US)

Correspondence Address: SAL ABRAHAM, 14305 CRYSTAL RIDGE DR., WATCHUNG, NJ, 07069, US

(73) Assignees: Mr. Vince Paternoster; Nutri Sport Pharmacal, Inc., Franklin, NJ (US)

(21) Appl. No.: 10/064,277

(22) Filed: Jun. 27, 2002

(51) Int. Cl. A61K 31/58; A61K 31/573

(52) U.S. Cl. 514/178; 514/172

(57) ABSTRACT

This invention relates to a method of increasing the anabolic/androgenic hormone 4-hydroxy-19-nortestosterone in humans by administering an effective amount of at least one of the ester or ether derivatives of 4-hydroxy-19-norandrosterone.

### BACKGROUND OF THE INVENTION

This invention relates to a method of imparting an anabolic/androgenic effect in humans by administering an effective amount of at least one of the ester or ether derivatives of 4-hydroxy-19-norandrosterone. 4-hydroxy-19-norandrosterone behaves as a prohormone and converts into 4-hydroxy-19-nortestosterone. In men, the normal balance of sex steroids is characterized by a greater amount of androgens over estrogens. As men age, production of androgen levels decreases. This decrease in androgens causes a hormonal imbalance in favor of the predominant female hormone estrogen. When estrogen exceeds androgen levels in males a cascade of detrimental effects can take place for instance, decreased sperm count, low free testosterone levels, decreased muscle mass and decreased strength. The use of 4-hydroxy -19-norandrosterone is intended to gradually increase the anabolic/androgenic hormone 4-hydroxy-19-nortestosterone without exhibiting negative side effects associated with steroid hormones such as hepatotoxicity, supraphysiological surges in androgen blood concentrations, and down regulation of the hypothalamic pituitary testicular axis (HPTA).

Androgens convert primarily to estrogen and feed back on the hypothalamic-pituitary axis to induce gonadotropin-releasing hormone (GnRH) synthesis with increased luteinizing hormone (LH) secretion and reduced follicular stimulating hormone (FHS) production. Increased luteinizing hormone (LH) secretion leads to the production of more testosterone and androstenedione, which inhibits production of sex hormone binding globulin and increases free androgen. Although as men age, less total testosterone is produced, which causes a shift in the androgen to estrogen ratio resulting in greater levels of estrogen also known as “Male Andropause”.

The standard treatment for this condition is intramuscular injection and/or peroral administration of pharmaceutical androgens. Various testosterone esters in oil depot form have been utilized as intramuscular injection. These weekly injections of synthetic testosterone cause supraphysiological surges in androgen levels which then leave the body deficient in androgens until the next injection. These supraphysiological levels of testosterones readily convert to estrogen and dihydrotestosterone (DHT) which can lead to side effects such gynocomastia and benign prostrate hypertrophy (BPH). Another negative side effect is the possible down regulation of the hypothalamic pituitary testicular axis (HPTA) resulting in loss of natural testosterone production.

The oral route of synthetic androgen administration consists of testosterone derivatives that are 17 alpha-alkylated for enhanced oral bioavailability (i.e. Methyltestosterone). This alkylation allow the steroids to withstand the 17 beta-hydroxyl oxidation in the liver. This appears to alleviate the dosing and blood hormone problems as compared to injections but it can cause undue stress to the liver and increase the possibility of hepatotoxicity.

U.S. Pat. No. 5,880,117 to Patrick Arnold, relates a method of using the oral precursor hormone 4-androstenediol as a means of increasing testosterone levels in humans. This hormone represents an improvement in standard therapies, since 4-androstenediol does not seem to be open to aromatase in its initial state. The possibility that this compound will convert to an estrogen over an androgen is chemically unlikely. The end product testosterone however is still readily aromatized. The compound suggested in this patent does offer advantages over standard therapies in that the compound in question avoids a direct path of estrogen conversion and provides a less toxic peroral route of administration. The target hormone of replacement may still be less than ideal due to it's conversion to estrogen and dihydrotestosterone (DHT).

U.S. Pat. No. 6,011,027 to Patrick Arnold, relates a method of using the oral precursor hormone 19-nor-4-androstenediol as a means of increasing 19-nortestosterone levels in humans. This hormone represents an improvement in standard therapies, since 19-nor-4-androstenediol converts into 19-nortestosterone thereby imparting the same anabolic effect as testosterone with much less androgenic activity. The possibility that this compound will convert to an estrogen over an androgen is chemically unlikely. The end product 19-nortestosterone also exhibits much less aromatization than testosterone. The compound suggested in this patent does offer advantages over standard therapies in that the compound in question avoids a direct path of estrogen conversion and provides a less toxic peroral route of administration. The target hormone of replacement may still be less than ideal due to it's poor oral absorption and bioavailability.

U.S. Pat. No. 6,242,436 to William Llewellyn, relates a method of using the oral precursor hormones 5alpha-androstanediol or 5alpha-androstanedione as a means of increasing dihydrotestosterone levels in humans. Dihydrotestosterone (DHT) is a more potent form of testosterone, shown to be roughly three to four times more active in the human body in comparison. This hormone represents an improvement, since 5alpha-androstanediol or 5alpha-androstanedione are natural, non-toxic, and quickly metabolized to active form after oral administration and unable to be aromatized into estrogens due to their structure. The compound suggested in this patent does offer advantages over standard therapies in that the compound in question avoids a direct path of estrogen conversion and provides a less toxic peroral route of administration. However, dihydrotestosterone (DHT) is not the most ideal form of testosterone due to it's causative relationship with benign prostrate hypertrophy (BPH) and other androgenic side effects.

U.S. patent application Ser. No. 10/063,415 and Ser. No. 10/064027, by Sal Abraham, discloses a method of effectively increasing natural testosterone and 4-hydroxytestosterone levels in humans by the oral administration of the ester and ether derivatives of 4-hydroxy-4-androstenedione and 4-hydroxytestosterone. U.S. patent application Ser. No. 10/063,415 demonstrates 4-hydroxy-4-androstenedione's ability lower estrogen and thus stimulate testosterone for the promotion of the variables associated with athletic function thereby enhancing physical performance. U.S. patent application Ser. No. 10/064027, discloses 4-hydroxytestosterone as an natural anabolic/androgenic metabolite of 4-hydroxy-4-androstenedione, which is an analog of the naturally occurring dietary supplement 4-androstenedione. To the best of my knowledge, the use of 4-hydroxytestosterone and 4-hydroxy-4-androstenedione for the regulation of athletic function as a dietary supplement has never been disclosed. The fact that 4-hydroxytestosterone is naturally made in the body during the metabolism of 4-hydroxy-4-androstenedione is not widely known and is justification for its use as a dietary supplement. 4-hydroxy-4-androstenedione can also be sold as a dietary supplement since it is an analog of the naturally occurring dietary supplement 4-androstenedione although this to is not widely known or understood. The esters and ethers of 4-hydroxy-4-androstenedione and 4-hydroxytestosterone impart an anabolic/androgenic effect and represent a novel and unobvious improvement in the regulation of athletic function as a dietary supplement. Although 4-hydroxy-19-nortestosterone represents still a further improvement over standard anabolic/androgenic therapies and represents a novel and unobvious improvement in male androgen replacement. To the best of my knowledge, the use of 4-hydroxy-19-norandrosterone or other hydroxylated 19-nor prohormones for the promotion of 4-hydroxy -19-nortestosterone levels in humans has never been previously disclosed.

### SUMMARY OF INVENTION

Standard therapy for male androgen deficiency includes synthetic exogenous administration of testosterone. However these therapies result in the aromatization of the target hormone and put undue stress upon the liver. U.S. Pat. No. 5,880,117 and U.S. Pat. No. 6,242,436 attempt to correct some of these problems by the peroral administration of androgen precursors. Now while the active androgen precursor is unable to aromatize, the target hormone of U.S. Pat. No. 5,880,117 can produce substantial conversion to estrogen and dihydrotestosterone (DHT) while the target hormone of U.S. Pat. No. 6,242,436 is directly converted to dihydrotestosterone (DHT). The problem of the present invention is to provide a compound that imparts an anabolic/androgenic effect without causing hepatotoxicity, supraphysiological surges in androgen blood concentrations, and down regulation of the hypothalamic pituitary testicular axis (HPTA). According to the invention these problems are solved by the use of the 4-hydroxy-19-norandrosterone. The use of this compound involves administrating an effective amount of at least one of the ester or ether derivatives of 4-hydroxy-19-norandrosterone in order to increase the anabolic/androgenic hormone 4-hydroxy-19-nortestosterone in humans with none of the previously mentioned negative side effects.

### DETAILED DESCRIPTION

The chemical term 4-hydroxy-19-norandrosterone refers to the five isomers: 4(3) alpha-dihydroxy-5alpha-estran-17-one, 4(3)alpha-dihydroxy-5beta-estran-17-one, 4(3)beta-dihydroxy-5beta-estran-17-one, 4(3)beta-dihydroxy-5alpha-estran-17one and 4(3)beta-dihydroxy-estran-17-one. This invention concerns all isomer forms of 4-hydroxy-19-norandrosterone. 4-hydroxy-19-norandrosterone is a naturally occurring hormone and metabolite of the naturally occurring hormone 19nortestosterone. Viable chemical esters of this compound include methyl, ethyl, propyl, butyl, and cyclohexyl carbonate as well as, acetate, heptanoate, decanoate, hemisuccinate, and benzoate. Viable chemical ethers include tetrahydropyranyl (THP), cyclopentyl (CPT), cyclohexyl and tetrahydrofuranyl (THF) ethers. This invention concerns all of the ester and ether isomer forms of 4(3)alpha-dihydroxy-5alpha-estran-17-one, 4(3)alpha-dihydroxy-5beta-estran-17-one, 4(3)beta-dihydroxy-5beta-estran-17-one, 4(3)beta-dihydroxy-5alpha-estran-17-one and 4(3)beta-dihydroxy-estran-17-one. The previous examples of various esters and ethers are presented by way of illustration only. It should be understood that this invention is not construed as limited in scope by the details contained therein, as it is apparent to those skilled in the art that modifications in materials and methods can be made without deviating from the scope of the invention. For example, 4-hydroxtylated forms 19-norandrostenedione or 19-norandrostenediol and other 4-hydroxtylated 19-nortestosterone metabolites with a reversible conversion pathway may be utilized for similar outcomes.

Recent studies by Le Bizec, B. et al. Steroids. 2002 February;67(2):105-10 demonstrates the endogenous production of 19-nortestosterone in man (ie. 2 ng/ml) 19-nortestosterone is chemically close to testosterone. This chemical difference imparts a noticeable differentiation between these two hormones. Testosterone is considered an androgenic/anabolic hormone while 19-nortestosterone is considered primarily anabolic, since 19-nortestosterone is missing the axial methyl group C19 (carbon 19) stemming off of C10 (carbon 10). 19-nortestosterone posses approximately the same anabolic effect as testosterone without the androgenic or male virilization effects (facial hair growth, body hair growth, male pattern baldness, and lowering of voice pitch). Testosterone is the major naturally occurring androgenic hormone produced by the interstitial (Leydig) cells of the testes in response to stimulation by the luteinizing hormone of the anterior pituitary. It regulates gonadotropic secretion and wolffian duct differentiation, and stimulates skeletal muscle. It is also responsible for other male characteristics and spermatogenesis after its conversion to dihydrotestosterone.

4-hydroxy-19-nortestosterone (4,17beta-Dihydroxyestr-4-en-3-one) or Oxabolone is a naturally occurring anabolic/androgenic 4-hydroxylated form of 19-nortestosterone. Steroid hydroxylation at C4 (carbon 4) has been demonstrated to occur in nature by Ding, X . et al. Arch. Biochem. Biophys . 315, 454-459. This is reaction is important in order to justify the metabolic pathway of 19-nortestosterone to 4-hydroxy-19-nortestosterone. Previous research by Waxman, D J. et al. Arch Biochem Biophys 1991 October;290(1):160-6 has demonstrated that individual human cytochrome P450 enzymes can hydroxylate endogenous steroid hormones with a high degree of stereospecificity and regioselectivity and that some, but not all of the human cytochromes exhibit metabolite profiles similar to their rodent counterparts.

Additional research by Le Bizec, B. et al. J Chromatogr B Biomed Sci Appl. 1999 February 19;723(1-2):157-72 has also shown 19-norandrosterone (3-Hydroxyestran-17one) to have an endogenous pathway in humans. Since steroid hydroxylation at C4 (carbon 4) has been demonstrated to occur in nature by Ding, X. et al. this justifies the metabolic pathway of 19-norandrosterone to 4-hydroxy-19-norandrosterone. Research by Raeside, J I. et al. J Steroid Biochem Mol Biol 1992 July;42(6):637-41 further supports this pathway. Both 11 beta and 6 beta-hydroxylated derivatives of 19-Norandrostenedione were found in porcine Leydig cells and a third product (11-oxo-19-Nor androstenedione) was tentatively identified. The profile of radioactive metabolites from [3H]19-Nortestosterone also favors the view of a capacity for hydroxylation of 19-norandrogens by porcine Leydig cells. Clouet, AS. et al. Analyst 1998 December;123(12):2489-92 has demonstrated in vivo metabolism in calf hepatocyte cultures of 19-nortestosterone. 19-Norepitestosterone, the main in vivo metabolite, was identified in vitro. However, the main in vitro metabolites were mostly in an oxidized form (4-estrene-3,17-dione, hydroxy-4-estrene-3,17-dione).

Human research performed by Poon, G K. et al. J Chromatogr 1991 April 19;565(1-2):75-88 demonstrated a similar steroid C4 hydroxylation pathway. This study demonstrated the pathway of 4-hydroxy-4-androstenedione to 4-hydroxytestosterone. 4-hydroxy-19-norandrosterone should behave as a prohormone and thus increase 4-hydroxy-19-nortestosterone levels in humans. 4-hydroxy-19-nortestosterone is an active anabolic/androgenic hormone that represents an improvement in previous 19-nor androgen hormone therapies due to no progestational activity, imparting anti-estrogenic effects, and possessing a very high anabolic to androgenic ratio. 19-nor androgens are know to have progestational activity and thus send negative feedback to the hypothalamic pituitary testicular axis (HPTA) causing a cessation of natural testosterone production. 19-nor androgens are also known to have less conversion into estrogen than testosterone but may still cause elevated estrogen levels. 4-hydroxy-19-nortestosterone possess no progestational activity and therefore should not negatively effect the hypothalamic pituitary testicular axis (HPTA). 4-hydroxy-19-nortestosterone cannot directly convert into estrogen and coupled with the anti-estrogenic effect should actually decrease circulating estrogen levels. The administration of an effective amount of the esters and/or ethers of 4-hydroxy-19-norandrosterone increases the anabolic/androgenic hormone 4-hydroxy-19-nortestosterone and thus represents a novel and unobvious improvement in male androgen replacement. 4-hydroxy-19-nortestosterone exhibits further improvements over other anabolic/androgenic hormones wherein its ability to impart a mild androgenic effect with a potent anabolic effect without causing hepatotoxicity, supraphysiological surges in androgen blood concentrations, and down regulation of the hypothalamic pituitary testicular axis (HPTA). 4-hydroxy-19-norandrosterone represents still a further improvement over standard anabolic/androgenic therapies which can consist of wasting syndromes, male androgen replacement, and when appropriate athletic function.

U.S. Pat. No. 3,060,201 to Bruno Camerino, relates a method for chemical manufacturing of 4-hydroxy-17alpha-methyl-3-keto-delta4-steroids of androstane and 19-nor-androstanes series and esters thereof. There are ten examples of various esters of 4-hydroxy-17alpha-methyl-testosterone, one example of 4,11beta-dihydroxy-17-alpha-methyltestosterone, and one example of 4-hydroxy-17alpha-methyl-19-nortestosterone. This patent discusses various medical uses associated with the retention of muscle and bone mass. It then discloses 4-hydroxy-17alpha-methyl-testosterone is devoid of progestational and sodium retaining activity. The 4-hydroxy-17alpha-methyl-19-nortestosterone is also devoid of progestational activity but also posses anti-estrogenic effects. 4-hydroxy-17alpha-methyl-19-nortestosterone myotrophic activity is noted to be twenty seven times higher than 17alpha-methyltestosterone.

Animal research performed by Gardi R, Falconi G, Pedrali C, Vitali R, Ercoli A (Steroids) 1972 May 19:5 639-47 demonstrates the androgenic and myogenic activity of orally administered ether modified alkyl androstan-17beta-yl mixed acetyls. The chemical addition of ethers and esters are designed to enhance oral absorption and bioavailability. The chemical addition of various ethers to the hydroxyl portion of the molecule, such as tetrahydropyranyl, tetrahydrofuranyl, cyclopentyl, or cyclohexyl ether, enables the compound to become lipophilic or fat soluble. This lipophilicity allows the compound to be absorbed via the lymphatic system and bypass the first-pass through the liver allowing more of the active compound to enter the bloodstream.

After an extensive review of the scientific literature regarding the ability of 4-hydroxy-19-norandrosterone to convert into 4-hydroxy-19-nortestosterone, it then became the focus of this invention that the esters and ethers of 4-hydroxy-19-norandrosterone could be administrated perorally as an effective means of imparting an anabolic/androgenic effect in humans without causing any of the previously mentioned negative side effects. The oral daily doses can be between 20 to 2000 mg., but preferably 100 to 600 mg. The preferred daily dosing schedule should be divided into 3-6 sub dose applications per day in order maintain adequate blood hormone concentrations. In addition to peroral use, the esters and ethers of 4-hydroxy-19-norandrosterone can be effectively administered by several other routes including transdermal, sublingual, and intranasal.

1. A method of increasing the 4-hydroxy-19-nortestosterone levels in humans by the administration of an effective amount of at least one of the ester or ether derivatives of 4-hydroxy-19-norandrosterone.

2. A method of claim 1, wherein said compound is 4,3alpha-dihydroxy-5alpha-estran -17-one.

3. A method of claim 1, wherein said compound is 4,3alpha-dihydroxy-5beta-estran -17-one.

4. A method of claim 1, wherein said compound is 4,3beta-dihydroxy-5beta-estran -17-one.

5. A method of claim 1, wherein said compound is 4,3beta-dihydroxy-5alpha-estran -17-one.

6. A method of claim 1, wherein said compound is 4,3beta-dihydroxy-estran -17-one.

7. A method of claim 1, wherein said ester derivatives include acetate, heptanoate, decanoate, hemisuccinate, benzoate and propionate.

8. A method of claim 1, wherein said carbonate ester derivatives include methyl, ethyl, propyl, butyl, and cyclohexyl.

9. A method of claim 1, wherein said ether derivatives include tetrahydropyranyl, tetrahydrofuranyl, cyclopentyl, and cyclohexyl.

10. A method of claim 1, wherein said administration is peroral.

11. A method of claim 1, wherein said administration is selected from the group consisting of transdermal, sublingual, and intranasal.

12. A method of claim 1, wherein said effective amount is a daily dosage of 20 mg to 2000 mg.
